(19) United States
(12) Patent Application Publication
(10) Pub. No.: US 2026/0306287 A1
(43) Pub. Date: Oct. 8, 2026

(54) MEDICAL BOOT

(71) Applicant: Osborn Medical Corporation, Centennial, CO (US)

(72) Inventors: William D. Davis, Denver, CO (US); Ian P. MacDonald, Denver, CO (US)

(21) Appl. No.: 19/478,303

(22) PCT Filed: Apr. 24, 2024
(86) PCT No.: PCT/US2024/025907

§ 371 Date: Oct. 24, 2025

Related U.S. Application Data
(60) Provisional application No. 63/498,038, filed on Apr. 25, 2023.

(51) Int. Cl. A61F 5/01

(52) U.S. Cl. CPC A61F 5/0195; A61F 5/0111

(57) ABSTRACT

A medical boot for a bedridden patient includes an exterior layer including a first fabric layer, a foam layer, and a second fabric layer laminated together and die cut into a shape having a posterior support portion shaped for wrapping around a lower leg, and a planar support portion extending from the posterior support portion. The planar support portion is shaped for wrapping around a foot. The medical boot includes one or more foam blocks for cushioning the lower leg and the foot. The medical boot further includes an interior layer attached to the exterior layer.

### CROSS-REFERENCE TO RELATED APPLICATIONS

This application claims priority to U.S. Patent Application No. 63/498,038 filed on Apr. 25, 2023, the disclosure of which is incorporated herein by reference in its entirety.

### BACKGROUND

Patients admitted to a healthcare facility such as a hospital, a nursing home, a rehabilitation center, a long-term care facility, and the like are sometimes bedridden for prolonged periods of time. Patients can experience health problems associated from lying down in one position for too long. For example, it can be desirable to improve blood circulation to prevent clotting, especially for patients who are diagnosed with vascular disease. However, patients who are bedridden for prolonged periods of time can experience vasoconstriction, which is the narrowing of blood vessels causing blood flow to be slowed or blocked.

Also, the feet of bedridden patients should be prevented from pointing downward or leaning to the side, which can lead to foot drop. Foot drop is a condition that may occur after lying in bed for a prolonged period of time without getting up or walking. Foot drop is the dropping of the forefoot due to weakness, damage to the peroneal nerve, or paralysis of the muscles in the anterior portion of the lower leg. It is characterized by the inability or difficulty in moving the ankle and toes upward and thereby leading to the improper rotation of the foot.

Further health problems experienced by patients who are bedridden for prolonged periods of time include skin ulcers that develop because of the heel rubbing against the bed sheets and/or pressure being placed on the heel while lying in a hospital bed.

### SUMMARY

In general terms, the present disclosure relates to a medical boot. In one possible configuration, the medical boot includes an exterior laminate layer that is die cut into a shape having a first portion shaped for wrapping around a lower leg, and a second portion extending from the first portion, the second portion shaped for wrapping around a foot. Various aspects are described in this disclosure, which include, but are not limited to, the following aspects.

One aspect relates to a medical boot for a bedridden patient, the medical boot comprising: an exterior layer including a first fabric layer, a foam layer, and a second fabric layer laminated together and die cut into a shape having: a posterior support portion shaped for wrapping around a lower leg; and a planar support portion extending from the posterior support portion, the planar support portion shaped for wrapping around a foot; one or more foam blocks positioned on the exterior layer; and an interior layer attached to the exterior layer.

Another aspect relates to a method of manufacturing a medical boot, the method comprising: forming an exterior layer by: laminating a first fabric layer, a foam layer, and a second fabric layer into a sheet; and die cutting the sheet into a shape having: a posterior support portion shaped for wrapping around a lower leg; and a planar support portion extending from the posterior support portion, the planar support portion shaped for wrapping around a foot; positioning one or more foam blocks on the exterior layer; attaching an interior layer to the exterior layer; and attaching one or more fasteners to the exterior layer, the one or more fasteners configured to attach to the first and second fabric layers of the exterior layer for removably wrapping the medical boot around the lower leg and the foot.

A variety of additional aspects will be set forth in the description that follows. The aspects can relate to individual features and to combination of features. It is to be understood that both the foregoing general description and the following detailed description are exemplary and explanatory only and are not restrictive of the broad inventive concepts upon which the embodiments disclosed herein are based.

### DESCRIPTION OF THE FIGURES

The following drawing figures, which form a part of this application, are illustrative of the described technology and are not meant to limit the scope of the disclosure in any manner.

FIG. 1 is a front isometric view of an example of a medical boot for a bedridden patient, the medical boot shown in an assembled state.

FIG. 2 is a rear isometric view of the medical boot of FIG. 1.

FIG. 3 is a side view of the medical boot of FIG. 1.

FIG. 4 is a front view of the medical boot of FIG. 1.

FIG. 5 is a rear view of the medical boot of FIG. 1.

FIG. 6 is a bottom view of the medical boot of FIG. 1.

FIG. 7 is a top view of the medical boot of FIG. 1.

FIG. 8 is an interior view of the medical boot of FIG. 1 in a disassembled state.

FIG. 9 is an exterior view of the medical boot of FIG. 1 in the disassembled state.

FIG. 10 is an exploded isometric view of the medical boot of FIG. 1.

FIG. 11 is an isometric view of an exterior layer of the medical boot of FIG. 1.

FIG. 12 is an elevated side view of the exterior laminate layer of FIG. 11 shown from a perspective facing a proximal end thereof.

FIG. 13 is a detailed view of the exterior layer show in FIG. 12.

FIG. 14 is a cross-sectional view of the exterior layer of FIG. 11.

FIG. 15 is a top view of the exterior layer of FIG. 11 showing exemplary dimensions.

FIG. 16 schematically illustrates an example of a method of manufacturing the medical boot of FIG. 1.

### DETAILED DESCRIPTION

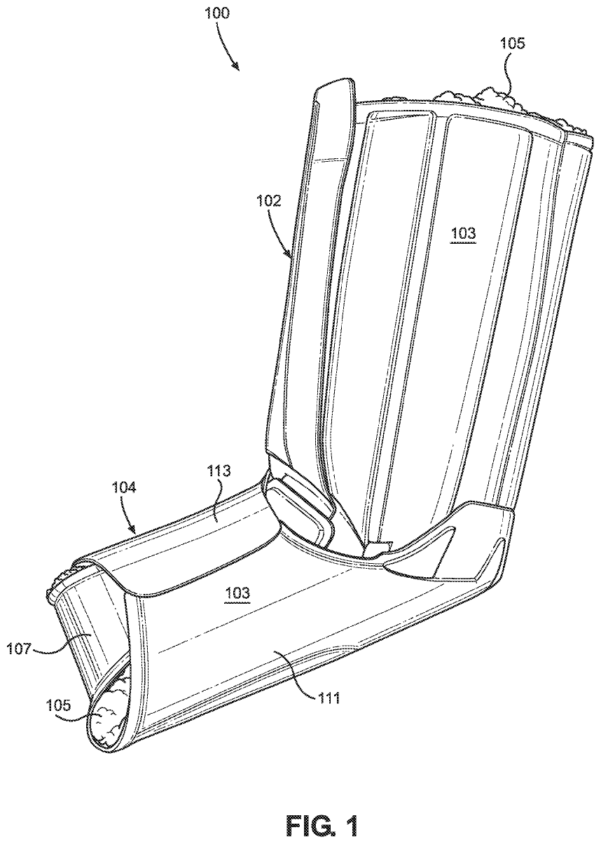

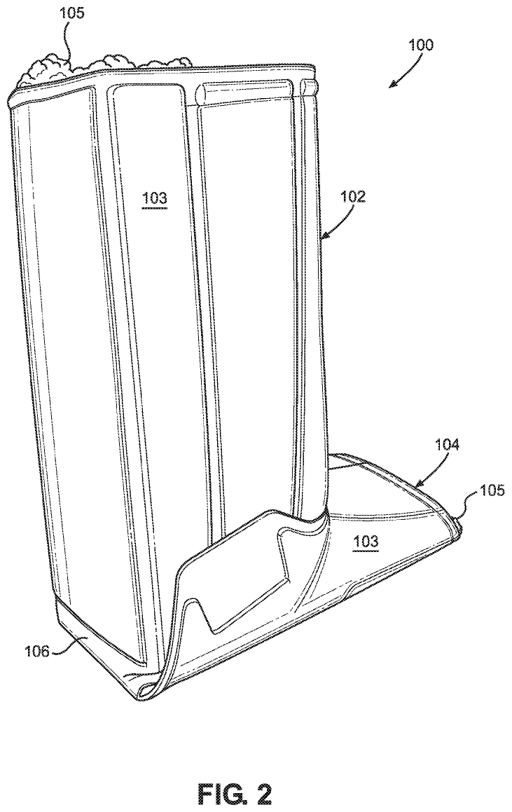

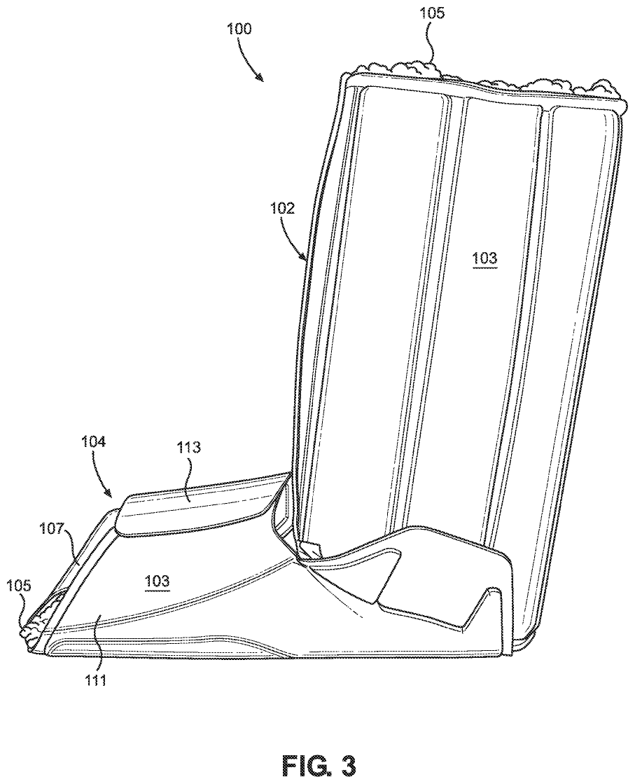

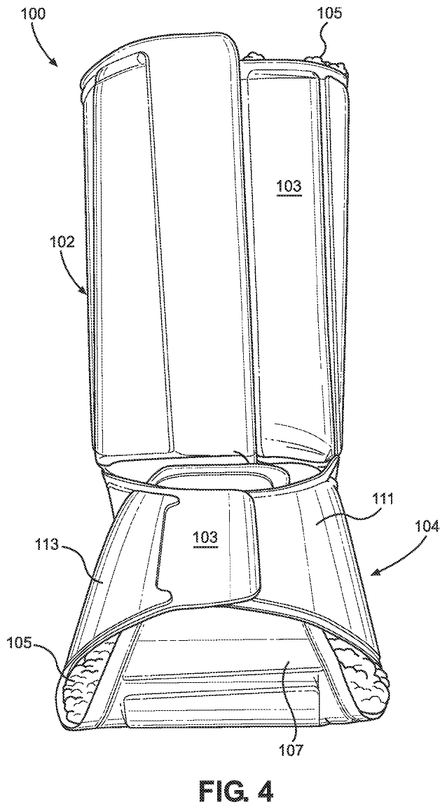

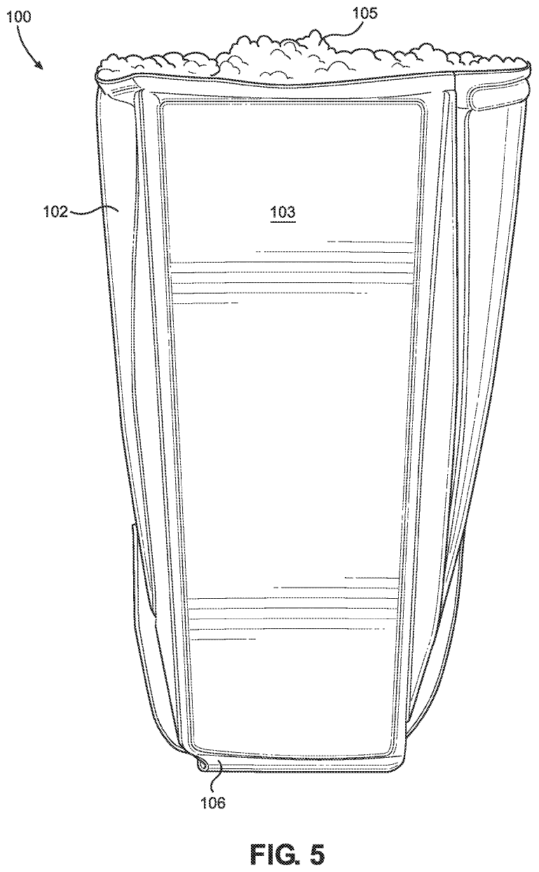

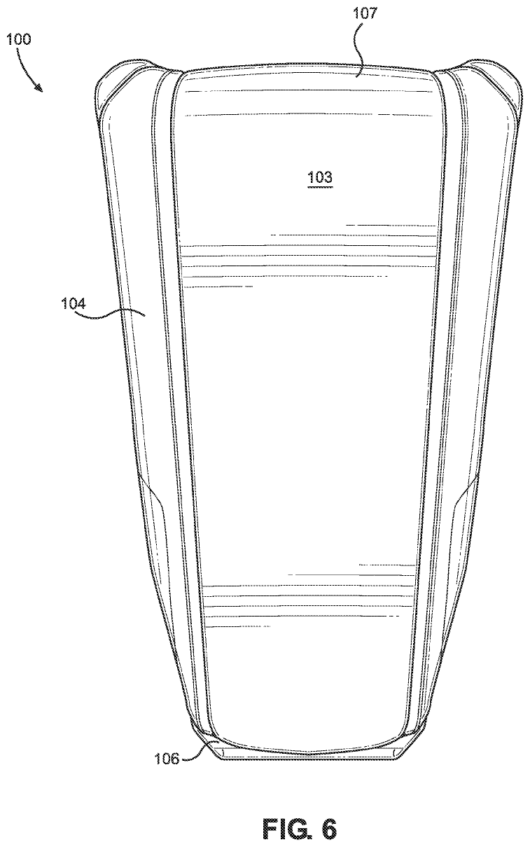

FIGS. 1-7 are respectively front isometric, rear isometric, side, front, rear, bottom, and top views of an example of a medical boot 100 for a bedridden patient. FIGS. 1-7 show the medical boot 100 in an assembled state such as when worn by a patient. The medical boot 100 can be worn by the patient when admitted to a healthcare facility such as a hospital, a nursing home, a rehabilitation center, a long-term care facility, and the like. Also, the medical boot 100 can also be worn by the patient when bedridden in their own home.

The medical boot 100 provides support for the foot of the patient by off-loading weight from the heel of the patient's foot which can help protect against pressure ulcers formed on the skin by elevating the heel relative to the bed. The medical boot 100 also helps to prevent foot drop by supporting the foot in a preferred position such as by keeping the foot in an upright position. This prevents the foot from falling over to one side or from pointing toward the foot of the bed while the patient remains lying down. The medical boot 100 can also be used to provide improved cushioning and to keep the patient's foot warm to improve blood circulation.

As shown in FIGS. 1-7, the medical boot 100 includes a posterior support portion 102 that is configured to wrap around a lower leg of the bedridden patient. The medical boot 100 also includes a planar support portion 104 that extends from the posterior support portion 102. The planar support portion is configured to wrap around a foot of the bedridden patient. The posterior support portion 102 and the planar support portion 104 are integral with one another.

The posterior support portion 102 covers a part of the lower leg of the patient including at least a portion of the calf and ankle. The posterior support portion 102 includes one or more foam pieces that elevate the heel of the patient's foot when the patient is lying on the patient's back to reduce pressure on the patient's heel, and thereby protect against pressure ulcers.

The planar support portion 104 of the medical boot 100 extends from the posterior support portion 102. The planar support portion 104 can pivot about a hinge 106 with respect to the posterior support portion 102. The hinge 106 integrally connects the planar support portion 104 and the posterior support portion 102. The planar support portion 104 covers the foot to protect against pressure ulcers and foot drop, and keeps the foot warm.

The posterior support portion 102 and the planar support portion 104 each include foam pieces sandwiched between an exterior layer 103 and an interior layer 105. The exterior layer 103 includes loop material that attaches to hook material of hook-and-loop fasteners. In some examples, the loop material is nylon fabric. The interior layer 105 includes an insulating fabric. In some examples, the insulating fabric includes a fleece material. In some further examples, the insulating fabric includes a polyester fleece material.

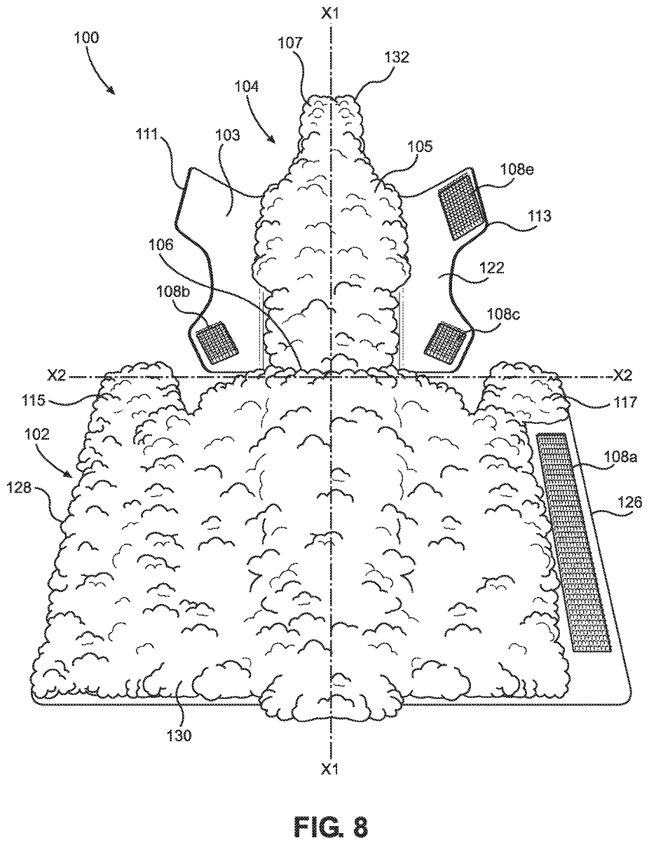

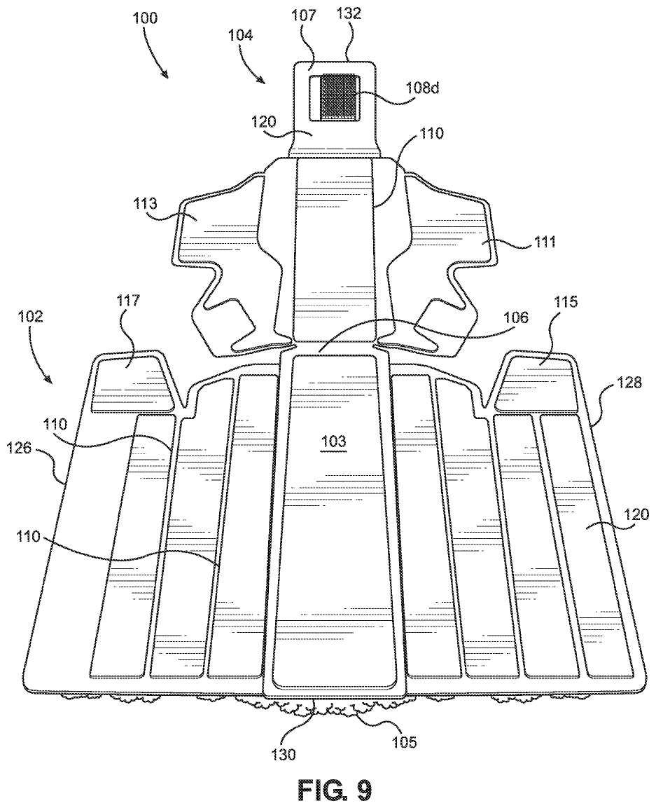

FIG. 8 is an interior view of the medical boot 100 in a disassembled state. FIG. 9 is an exterior view of the medical boot 100 in a disassembled state. As shown in FIGS. 8 and 9, the medical boot 100 continuously extends from a proximal end 130 of the posterior support portion 102 to a distal end 132 of the planar support portion 104 as a single assembly.

A plurality of fasteners 108 are attached to the exterior layer 103. In some examples, the fasteners 108 include hook fasteners. In some examples, the fasteners 108 are sewn onto the exterior layer 103. Additional means for attaching the fasteners 108 to the exterior layer 103 are contemplated. The fasteners 108 removably attach to the loop material included on the exterior layer 103 to wrap the medical boot 100 around the lower leg and foot of the patient.

To wrap the medical boot 100 around the lower leg of the patient, the posterior support portion 102 includes a fastener 108a attached to an interior surface layer 122 on a first side 126 of the medical boot. The first side 126 can be pulled around a first axis X1 of the medical boot 100 such that the fastener 108a removably attaches to an exterior surface layer 120 on a second side 128 of the medical boot. The exterior layer 103 includes the exterior and interior surface layers 120, 122, which are made of a loop material such as nylon fabric. In this manner, the medical boot 100 can be secured around the lower leg of the patient.

The hinge 106 pivots about a second axis X2. The planar support portion 104 includes fasteners 108b, 108c on the interior surface layer 122 that attach to the exterior surface layer 120 on the posterior support portion 102. The fasteners 108b, 108c removably fix the planar support portion 104 to the posterior support portion 102 in a pivoted orientation. When in the pivoted orientation, planar support portion 104 is substantially perpendicular with respect to the posterior support portion 102 (see FIGS. 1-7).

The planar support portion 104 can be wrapped around the patient's foot as follows. The planar support portion 104 includes a tongue 107 that folds over a top of the patient's foot. The tongue 107 overlaps tabs 115, 117 that extend from the posterior support portion 102. The tabs 115, 117 are shaped and sized to cover the anterior side of the ankle.

The tongue 107 includes a fastener 108d attached to the exterior surface layer 120 of the exterior layer 103. A first wing 111 of the planar support portion 104 can be positioned over the tongue 107 such that the fastener 108d attaches the interior surface layer 122 of the exterior layer 103 of the first wing 111 to the tongue 107.

A second wing 113 of the planar support portion 104 includes a fastener 108e. The second wing 113 can be placed over the first wing 111 such that the fastener 108e attaches to the exterior surface layer 120 of the exterior layer 103.

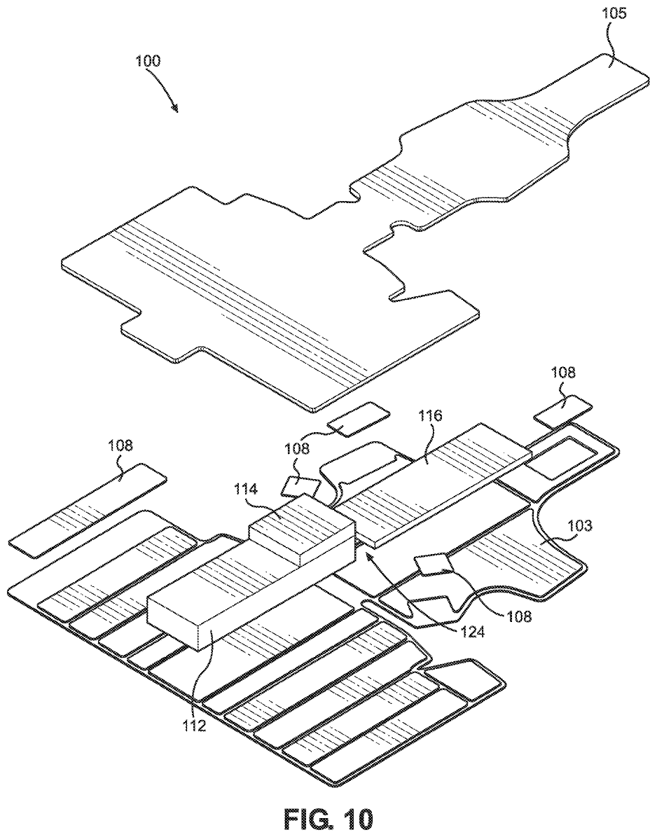

FIG. 10 is an exploded isometric view of the medical boot 100. As shown in FIG. 10, one or more foam blocks for cushioning the lower leg and the foot are positioned between the exterior layer 103 and the interior layer 105. The one or more foam blocks can include a first foam block 112 positioned in the posterior support portion 102. The one or more foam blocks can further include a second foam block 114 positioned on the first foam block 112.

The first foam block 112 has a first density. The second foam block 114 has a second density different from the first density of the first foam block 112. For example, the second density can be lower than the first density. In some examples, the first foam block 112 includes a polyurethane foam. In some examples, the second foam block 114 includes a memory foam.

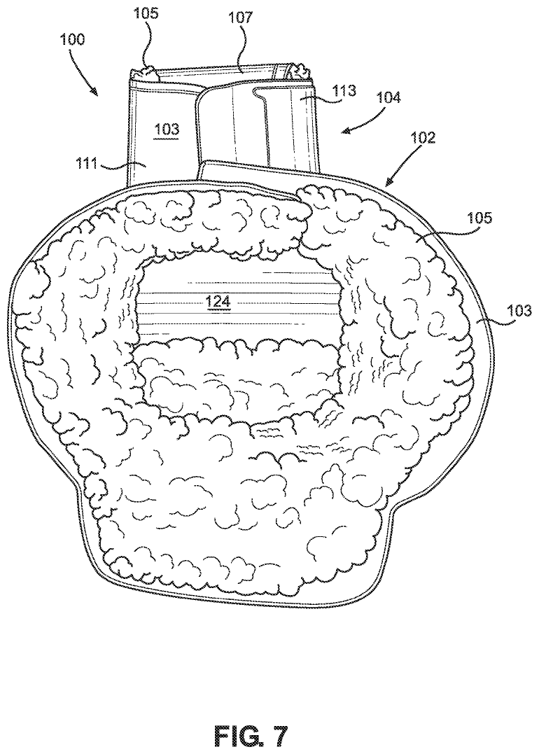

The first and second foam blocks 112, 114 form an L-shape cross-sectional profile. The L-shape cross-sectional profile elevates the heel of the patient's foot to reduce pressure on the heel to protect against pressure ulcers. For example, the calcaneal tendon, which is a tendon at the back of the lower leg (also known as the Achilles tendon), can be positioned on top of the second foam block 114 such that the heel of the foot floats in a space 124 (see FIG. 7) defined where the planar support portion 104 pivots with respect to the posterior support portion 102.

As further shown in FIG. 10, the one or more foam blocks further include a third foam block 116 positioned in the planar support portion 104. When the planar support portion 104 pivots about the hinge 106 such as when the medical boot 100 in the assembled state shown in FIGS. 1-7, the third foam block 116 is substantially perpendicular to the first and second foam blocks 112, 114. The third foam block 116 cushions the bottom of the patient's foot. In some examples, the third foam block 116 includes a rubber foam. As shown in FIG. 10, the interior layer 105 attaches to the exterior layer 103 to secure the first, second, and third foam blocks 112, 114, 116 between the exterior layer 103 and the interior layer 105.

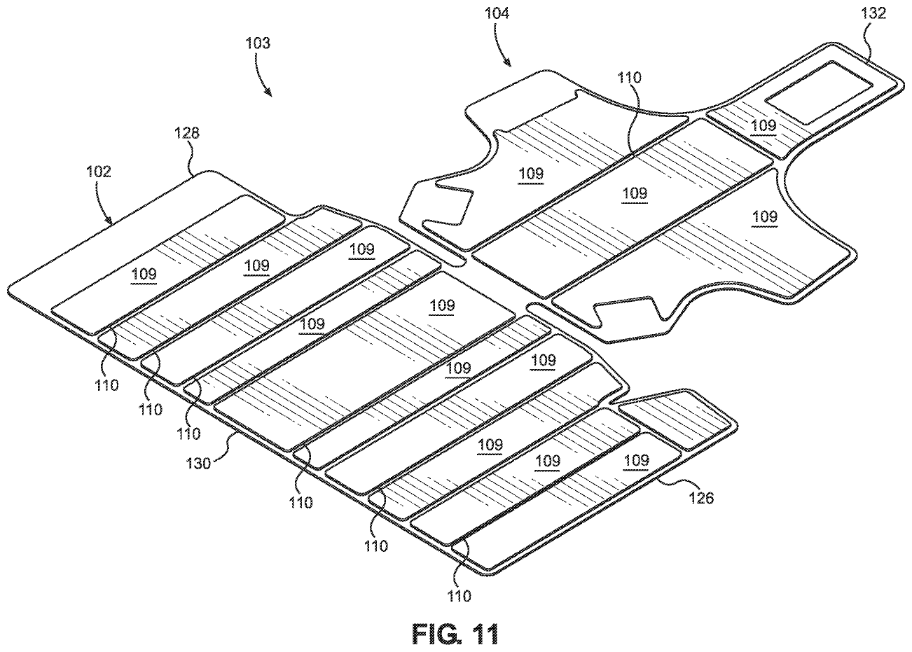

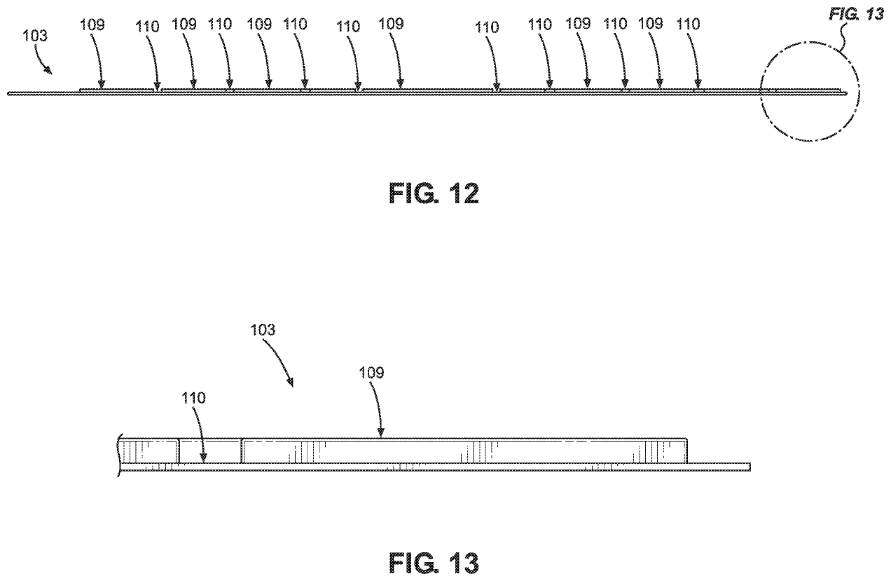

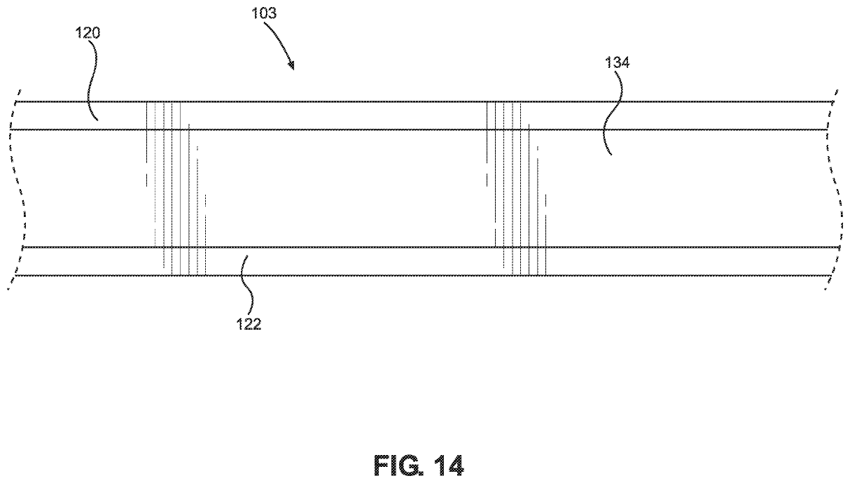

FIG. 11 is an isometric view of the exterior layer 103. FIG. 12 is an elevated side view of the exterior layer 103 shown from a perspective facing the proximal end 130. FIG. 13 is a detailed view of exterior layer 103 shown in FIG. 12. FIG. 14 is a cross-sectional view of the exterior layer 103. Referring now to FIGS. 11-14, the exterior layer 103 includes multiple layers that are laminated together using one or more of heat, pressure, and adhesives.

In the example of FIGS. 11-14, the exterior layer 103 includes the exterior surface layer 120 and the interior surface layer 122. A foam layer 134 is positioned between the exterior surface layer 120 and the interior surface layer 122. The exterior surface layer 120, the foam layer 134, and the interior surface layer 122 are laminated together into a single sheet of material, which is then die cut into a shape that includes the posterior support portion 102 and the planar support portion 104 extending from the posterior support portion (see FIGS. 11 and 12).

As described above, the exterior and interior surface layers 120, 122 include a loop material such as a fabric that attaches to the fasteners 108. In some examples, the exterior surface layer 120 and the interior surface layer 122 include nylon fabric. The exterior surface layer 120 and the interior surface layer 122 are first and second fabric layers.

In some examples, the foam layer 134 includes an ethylene-vinyl acetate (EVA) closed cell foam. The exterior surface layer 120, the foam layer 134, and the interior surface layer 122 are compressed together to form compression lines 110. As shown in FIG. 13, the compression lines 110 can have a depth of about 1/32 inches. As further shown in FIG. 13, elevated portions 109 can extend above the compression lines 110 by about ⅛ inches.

The foam layer 134 can improve the strength of the exterior layer 103. The compression lines 110 can facilitate wrapping the posterior support portion 102 around the lower leg, and wrapping the planar support portion 104 around the foot. Additionally, by laminating together the exterior surface layer 120, the foam layer 134, and the interior surface layer 122, an interior area defined between the first side 126, the second side 128, the proximal end 130, and the distal end 132 of the medical boot 100 is seamless.

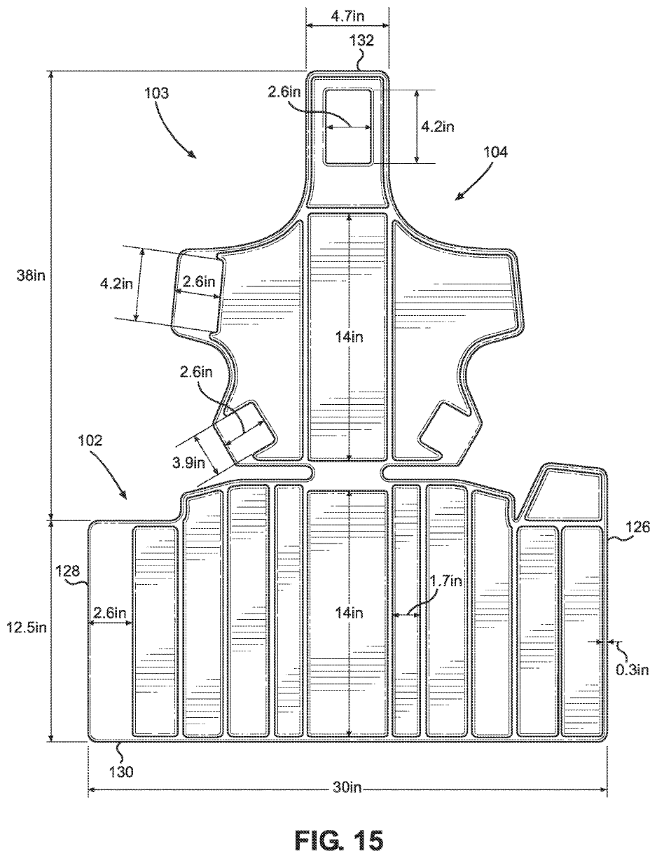

FIG. 15 is a top view of the exterior layer 103 showing exemplary dimensions. For example, the posterior support portion 102 can extend between the first and second sides 126, 128 by a width of about 30 inches. Also, the medical boot 100 can extend between the proximal end 130 and the distal end 132 by a length of about 38 inches. The dimensions shown in FIG. 15 are provided by way of illustrative example such that it is contemplated that the dimensions of the medical boot 100 may vary from the dimensions shown in FIG. 15.

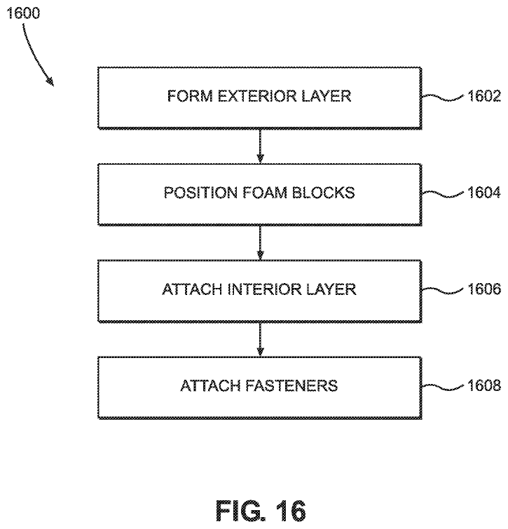

FIG. 16 schematically illustrates an example of a method 1600 of manufacturing the medical boot 100. The method 1600 can reduce costs for manufacturing the medical boot 100. Also, the method 1600 can reduce the time for manufacturing the medical boot 100.

The method 1600 includes an operation 1602 of forming the exterior layer 103. Operation 1602 can include laminating a first fabric layer (e.g., the exterior surface layer 120), the foam layer 134, and a second fabric layer (e.g., the interior surface layer 122) into a sheet. Operation 1602 can include laminating the first fabric layer, the foam layer 134, and the second fabric layer into the sheet using one or more of heat, pressure, and adhesives.

Operation 1602 can include applying pressure to the first fabric layer, the foam layer 134, and the second fabric layer to form the compression lines 110. Operation 1602 can include die cutting the sheet into a shape having the posterior support portion 102, and the planar support portion 104 that continuously extends from the posterior support portion 102.

Performance of operation 1602 results in the exterior layer 103 having an interior area that is seamless between the first side 126, the second side 128, the proximal end 130, and the distal end 132. Additionally, the exterior layer 103 includes the first and second fabric layers (e.g., the exterior surface layer 120 and the interior surface layer 122) that are made of loop material such as nylon fabric or similar types of fabric that attaches to the fastener 108.

Next, the method 1600 includes an operation 1604 of positioning one or more foam blocks on the exterior layer 103. Operation 1604 can include positioning the first foam block 112 in the posterior support portion 102. Operation 1604 can further include positioning the second foam block 114 on the first foam block 112. As described above, the first and second foam blocks 112, 114 provide an L-shape cross-sectional profile. Operation 1604 can further include positioning the third foam block 116 in the planar support portion 104.

Next, the method 1600 includes an operation 1606 of attaching the interior layer 105 to the exterior layer 103. The interior layer 105 can be made of an insulating fabric such as fleece. In some examples, the interior layer 105 is made of polyester fleece. In some examples, operation 1606 includes sewing the interior layer 105 onto the exterior layer 103. By attaching the interior layer 105 to the exterior layer 103, the one or more foam blocks are positioned between the exterior layer 103 and the interior layer 105 of the medical boot 100.

Next, the method 1600 includes an operation 1608 of attaching the fasteners 108 to the exterior layer 103. As described above, the fasteners 108 can include fasteners that attach to the first and second fabric layers (e.g., the exterior and interior surface layers 120, 122) of the exterior layer 103 for removably wrapping the medical boot around the lower leg and the foot. Operation 1608 can include sewing the fasteners 108 onto the exterior layer 103.

The various embodiments described above are provided by way of illustration only and should not be construed to be limiting in any way. Various modifications can be made to the embodiments described above without departing from the true spirit and scope of the disclosure.

What is claimed is:

1. A medical boot for a bedridden patient, the medical boot comprising:
an exterior layer including a first fabric layer, a foam layer, and a second fabric layer laminated together and die cut into a shape having:
a posterior support portion shaped for wrapping around a lower leg; and
a planar support portion extending from the posterior support portion, the planar support portion shaped for wrapping around a foot;
one or more foam blocks positioned on the exterior layer; and
an interior layer attached to the exterior layer.

2. The medical boot of claim 1, wherein the first and second fabric layers are made of a loop material.

3. The medical boot of claim 2, wherein the exterior layer further includes fasteners that removably attach to the loop material.

4. The medical boot of claim 1, wherein the exterior layer includes compression lines that facilitate wrapping the posterior support portion around the lower leg, and that facilitate wrapping the planar support portion around the foot.

5. The medical boot of claim 1, wherein the exterior layer includes an interior area between a first side, a second side, a proximal end, and a distal end, and the interior area is seamless.

6. The medical boot of claim 1, wherein the one or more foam blocks include:
a first foam block positioned in the posterior support portion, the first foam block having a first density; and
a second foam block positioned on the first foam block, the second foam block having a second density lower than the first density.

7. The medical boot of claim 6, wherein the first and second foam blocks form an L-shape cross-sectional profile that elevates a heel of the foot.

8. The medical boot of claim 6, wherein the one or more foam blocks further include:
a third foam block positioned in the planar support portion.

9. The medical boot of claim 1, wherein the interior layer is made of a fleece material.

10. The medical boot of claim 1, wherein the planar support portion continuously extends from the posterior support portion as a single assembly.

11. A method of manufacturing a medical boot, the method comprising:
forming an exterior layer by:
laminating a first fabric layer, a foam layer, and a second fabric layer into a sheet; and
die cutting the sheet into a shape having:
a posterior support portion shaped for wrapping around a lower leg; and
a planar support portion extending from the posterior support portion, the planar support portion shaped for wrapping around a foot;
positioning one or more foam blocks on the exterior layer;
attaching an interior layer to the exterior layer; and
attaching one or more fasteners to the exterior layer, the one or more fasteners configured to attach to the first and second fabric layers of the exterior layer for removably wrapping the medical boot around the lower leg and the foot.

12. The method of claim 11, wherein positioning the one or more foam blocks on the exterior layer includes:
positioning a first foam block in the posterior support portion; and
positioning a second foam block on the first foam block, the first and second foam blocks forming an L shape cross-sectional profile.

13. The method of claim 12, wherein the first foam block has a first density, and the second foam block has a second density that is less than the first density.

14. The method of claim 12, wherein positioning the one or more foam blocks on the exterior layer further includes:
positioning a third foam block in the planar support portion.

15. The method of claim 11, wherein laminating the first fabric layer, the foam layer, and the second fabric layer into the sheet produces compression lines that facilitate wrapping the medical boot around the lower leg and the foot.

16. The method of claim 11, wherein the exterior layer includes an interior area between a first side, a second side, a proximal end, and a distal end, and the interior area is seamless.

17. The method of claim 11, wherein the first and second fabric layers are made of a loop material, and the one or more fasteners are hook fasteners.

18. The method of claim 17, wherein the interior layer is made of a fleece material.

19. The method of claim 18, wherein the interior layer is sewn onto the exterior layer.

20. The method of claim 19, wherein the planar support portion continuously extends from the posterior support portion.
